(19) United States
(12) Patent Application Publication
(10) Pub. No.: US 2012/0136048 A1
(43) Pub. Date: May 31, 2012

(54) TOPICAL, PERIOCULAR, OR INTRAOCULAR USE OF TOCOTRIENOLS FOR THE TREATMENT OF OPHTHALMIC DISEASES

(76) Inventors: Guy M. Miller, Monte Sereno, CA (US); William D. Shrader, Belmont, CA (US); Viktoria Kheifets, Mountain View, CA (US)

(21) Appl. No.: 13/266,240

(22) PCT Filed: Apr. 27, 2010
(86) PCT No.: PCT/US10/32622

§ 371 Date: Jan. 18, 2012

(30) Foreign Application Priority Data
Apr. 28, 2009 (US) 61214760

(51) Int. Cl. A61K 31/353; A61P 27/04; A61P 25/00; A61P 25/16; A61P 25/08; A61P 25/14; A61P 27/06; C07D 311/72; C07F 9/655; C07D 405/12; A61P 27/02; A61P 25/28

(52) U.S. Cl. 514/456; 549/408; 549/410; 549/220; 546/282.7

(57) ABSTRACT

A method for preventing, reducing, ameliorating or treating ophthalmic disorders associated with neurodegenerative diseases or trauma, comprising the topical, periocular, or intraocular application of an ophthalmic formulation comprising a therapeutically effective amount of one or more ophthalmic agents selected from the group consisting of alpha-tocotrienol, beta-tocotrienol, gamma-tocotrienol, delta-tocotrienol, or esters or mixtures thereof is disclosed. Use of tocotrienols for the prevention, reduction, amelioration or treatment of ophthalmic disorders that are, or that are associated with, mitochondrial diseases is also discussed. Topical ophthalmic formulations comprising tocotrienols are also discussed.

### CROSS-REFERENCE TO RELATED APPLICATIONS

This application claims priority benefit of U.S. Provisional Patent Application No. 61/214,760, filed Apr. 28, 2009. The entire content of that application is hereby incorporated by reference herein in its entirety

### TECHNICAL FIELD

The present invention relates to a topical, periocular, or intraocular use of tocotrienols, tocotrienol esters or mixtures thereof, to prevent, reduce, ameliorate, or treat ophthalmic disorders or to stop the progression of or reverse the loss of vision in a patient in need of such treatment. The present invention relates to a topical, periocular, or intraocular use of one or more tocotrienols, tocotrienol esters or mixtures thereof, to prevent, reduce, ameliorate, or treat ophthalmic disorders or to stop the progression of or reverse the loss of vision associated with neurodegenerative diseases or trauma. The present invention relates to the topical, periocular, or intraocular use of tocotrienols, tocotrienol esters or mixtures thereof, to prevent, reduce, ameliorate, or treat ophthalmic disorders, or to stop the progression of or reverse the loss of vision associated with mitochondrial myopathies. The present invention relates to the topical, periocular, or intraocular use of one or more tocotrienols, tocotrienol esters or mixtures thereof to prevent, reduce, ameliorate, treat or reverse vision loss in patients suffering from chronic progressive external ophthalmoplegia (CPEO), dominant optic atrophy (DOA), or Leber's Hereditary Optic Neuropathy (LHON). The present invention relates to a topical, periocular, or intraocular formulation beneficial to a patient suffering from or at risk of ophthalmic disorders or vision loss, said formulation comprising a therapeutically effective amount of one or more agents selected from tocotrienols, tocotrienol esters or mixtures thereof.

### BACKGROUND OF THE INVENTION

Mitochondrial myopathies are a group of diseases caused by damage to the mitochondria—small, energy-producing structures that serve as the cells' “power plants.” Inherited changes in mitochondrial DNA can cause problems with growth, development, and function of the body's systems. These mutations disrupt the mitochondria's ability to efficiently generate energy for the cell, with the worst effects occurring in the organs with highest energy need. Although the health consequences of inherited mitochondrial DNA mutations vary widely, some frequently observed features include abnormalities involving the eyes and vision, including but not limited to visual loss and blindness, ptosis, ophthalmoplegia optic atrophy, acquired strabismus, and retinitis pigmentosa. (Kosmorsky, et al., Neurol. Clin. (1991) 9:147-61 and Biousse, V. et al., Curr. Opin. Neurol. (2003) 16 (1): 35-43).

Mitochondrial myopathies and associated disorders include but are not limited to Leber's Hereditary Optic Neuropathy (LHON), Dominant Optic Atrophy (DOA), Chronic Progressive External Ophthalmoplegia (CPEO), Spinocerebellar ataxia (SCA), also called Machado-Joseph disease, Leigh's Syndrome, Friedreich's ataxia (FRDA), Mitochondrial myopathy, Encephalopathy, Lactacidosis, and Stroke (MELAS); Myoclonic Epilepsy with Ragged Red Fibers (MERRF), Kearns-Sayre Syndrome (KSS), Co-Enzyme Q10 (CoQ10) Deficiency; Complex I deficiency, Complex II deficiency, Complex III deficiency, Complex IV deficiency, and Complex V deficiency. Many patients with mitochondrial myopathies including ataxia symptoms have eye movement abnormalities (especially slowed saccades, abnormal pursuit, and nystagmus), optic neuropathy (especially among patients with Friedrich's ataxia), and retinal degeneration (spinocerebellar ataxia); Gouw et al, Nature Genetics (1995) 10, 89-93.

Leber's Hereditary Optic Neuropathy (LHON) is characterized by blindness which occurs on average between 27 and 34 years of age; blindness can develop in both eyes simultaneously, or sequentially (one eye will develop blindness, followed by the other eye two months later on average). Autosomal Dominant Optic Atrophy (DOA) is the most common form of hereditary optic neuropathy, characterized by retinal ganglion cell degeneration leading to optic neuropathy. DOA presents in the first decade of life and manifests as progressive vision loss. In DOA retinal ganglion cells and the optic nerve degenerate by an unknown mechanism. The gene mutated in DOA, Optic Atrophy Type 1 (OPA1) is predominantly expressed in retinal ganglion cells of the retina and axons of the optic nerve. Zanna et al, Brain 2008 131(2):352-367.

Chronic Progressive External Ophthalmoplegia (CPEO) is a disorder characterized by slowly progressive paralysis of the extraocular muscles. Patients usually experience bilateral, symmetrical, progressive ptosis, followed by ophthalmoparesis months to years later. Ciliary and iris muscles are not involved. CPEO is the most frequent manifestation of mitochondrial myopathies. CPEO in association with mutations in mitochondrial DNA (mtDNA) may occur in the absence of any other clinical sign, but it is usually associated with skeletal muscle weakness.

Leigh's syndrome (also known as Leigh's disease or subacute necrotizing encephalomyelopathy) is one of many mitochondrial disorders. It is a progressive neurodegenerative disorder due to a wide variety of genetic mutations in mitochondrial DNA (mtDNA) or in nuclear DNA (gene SURF1 and some COX assembly factors). It is an inherited disorder that usually affects infants between the age of three months and two years, but, in rare cases, teenagers and adults as well. Some of the symptoms include loss of vision, and abnormal eye movements. Other symptoms include psychomotor delay/regression with superimposed signs of basal ganglia and brain stem dysfunction: ataxia, ophthalmoplegia, and dystonia.

Friedreich's ataxia (FRDA) is an autosomal recessive neurodegenerative and cardiodegenerative disorder caused by decreased levels of the protein frataxin. The disease causes the progressive loss of voluntary motor coordination (ataxia) and cardiac complications. Symptoms typically begin in childhood, and the disease progressively worsens as the patient grows older; patients eventually become wheelchair-bound due to motor disabilities. Some people with Friedreich's ataxia develop loss of visual acuity or changes in color vision. Most have jerky eye movements (nystagmus), but these movements by themselves do not necessarily interfere with vision.

Mitochondrial myopathy, Encephalopathy, Lactacidosis, and Stroke (MELAS) is a disease that can manifest itself in infants, children, or young adults. Ocular changes in MELAS syndrome have included reversible scotomata, ophthalmoplegia, and pigmentary retinopathy.

Kearns-Sayre Syndrome (KSS) is characterized by a triad of features including: (1) typical onset in persons younger than age 20 years; (2) chronic, progressive, external ophthalmoplegia; and (3) pigmentary degeneration of the retina. In addition, KSS may include cataracts.

Spinocerebellar ataxia (SCA), also called Machado-Joseph disease, is characterized by slowly progressive incoordination of gait and often associated with poor coordination of hands, speech, and eye movements. Nystagmus and macular degeneration are two characteristics of this disease. Gupta, S et al., Journal of Neurological Sciences (2008) 264: 173-176 have disclosed the diagnosis of spinocerebellar ataxia with vision loss secondary to retinal pigmentary dystrophy.

Yet another devastating syndrome resulting from a respiratory chain disorder is Co-Enzyme Q10 (CoQ10) Deficiency, the symptoms of which include encephalomyopathy, mental retardation, exercise intolerance, ragged-red fibers, and recurrent myoglobin in the urine. CoQ10 Deficiency has also been associated with eye movement symptoms.

Yet other syndromes, named overlap syndromes, combine the clinical features of different typical mitochondrial syndromes. One such syndrome characterized by clinical features of both myoclonus epilepsy with ragged-red fibers (MERRF) and Kearns-Sayre syndrome (KSS), and due to a mitochondrial DNA (mtDNA) mutation at nucleotide 3255 (G3255A) of the tRNA^{Leu(UUR)} gene has been described by Nishigaki, Y et al., Neuromuscular Disorders (2003) 13:334-340. This particular overlap syndrome manifests sensorineural deafness, atypical pigmentary retinopathy, myoclonus epilepsy, ptosis, ophthalmoparesis, migraine headaches, hypothyroidism, and testosterone insufficiency.

Glaucoma is part of a group of diseases of the optic nerve involving loss of retinal ganglion cells in a characteristic pattern of optic neuropathy. Raised intraocular pressure is a significant risk factor for developing glaucoma (above 22 mmHg). One person may develop nerve damage at a relatively low pressure, while another person may have high eye pressure for years and yet never develop damage. Untreated glaucoma leads to permanent damage of the optic nerve and resultant visual field loss, which can progress to blindness.

Glaucoma can be divided roughly into two main categories, “open angle” or chronic glaucoma and “closed angle” or acute glaucoma. Angle closure, acute glaucoma appears suddenly and often with painful side effects and so is usually diagnosed quickly, although damage and loss of vision can also occur very suddenly. Primary open-angle glaucoma (POAG) is a progressive disease leading to optic nerve damage and, ultimately, loss of vision. Glaucoma results in the neuronal degeneration of the retina and optic nerve head. Even with aggressive medical care and surgical treatment, the disease generally persists causing a gradual loss of retinal neurons (retinal ganglion cells (“RGCs”)), a decline of visual function, and ultimately blindness.

Diabetic retinopathy (DR) is a common complication of diabetes and a leading cause of legal blindness in working-age adults. The clinical hallmarks of DR include increased vascular permeability, leading to edema, and endothelial cell proliferation. Much of the research effort has been focused on vascular changes, but it is becoming apparent that other degenerative changes occur beyond the vascular cells of the retina. These include increased apoptosis, glial cell reactivity, microglial activation, and altered glutamate metabolism. When occurring together, these changes may be considered as neurodegenerative and could explain some of the functional deficits in vision that begin soon after the onset of diabetes.

Age-related macular degeneration (AMD) is a disease associated with aging that gradually destroys sharp, central vision. Central vision is needed for seeing objects clearly and for common daily tasks such as reading and driving. AMD affects the macula, the part of the eye that provides humans with the ability to see fine detail. AMD causes no pain. In some cases, AMD advances so slowly that people notice little change in their vision. In others, the disease progresses faster and may lead to a loss of vision or legal blindness in both eyes. AMD is a leading cause of vision loss in Americans 60 years of age and older. It occurs in two forms: wet and dry.

Other forms of macular degeneration (MD) sometimes covered under Juvenile Macular Degeneration (JMD) include Stargardt's disease, Best's vitelliform retinal dystrophy, Doyne's honeycomb retinal dystrophy, Malattia leventinese, Sorsby's fundus dystrophy, and Autosomal dominant hemorrhagic macular dystrophy. Stargardt's disease is the most common type of JMD. Symptoms typically develop in childhood or teen years. Symptoms include decline in visual acuity, drusen spots on the macula and scarring of the macula. Best's vitelliform retinal dystrophy, the second most common JMD, is usually a relatively mild form of macular degeneration. Its most distinctive symptom is an “egg yolk” large drusen spot on the macula at an early stage, which later breaks up into “scrambled egg” drusen.

Alzheimer's disease is a common progressive neurodegenerative disease that affects approximately 4 million people in the United States. In about one-third of Alzheimer's cases, there is a predominantly “visual” presentation in which symptoms of visual cortical dysfunction dominate. These patients usually present with vague complaints of poor vision, problems with way-finding, and problems reading.

Progressive Supranuclear Palsy (PSP) is a rare neurodegenerative disorder that combines an abnormality of voluntary eye movements with preserved vestibular ocular reflex movements, impaired postural reflexes with falling backwards, and Parkinsonism.

Parkinson Disease (PD) and other Parkinson-like diseases (called Parkinsonisms) frequently cause increasing vision problems as the illness progresses. As PD or a related disease progresses, many patients develop increasingly poor eyesight (functionally reduced visual acuity).

Patients with Amyotrophic Lateral Sclerosis (ALS) typically experience ocular abnormalities thought to be caused by dysfunction in the neural system that controls motor performance. Patients that have been on a ventilator for long periods may have a high frequency of ocular abnormalities, such as the inability to voluntary close the eyes or complete ocular paralysis (ophthalmoplegia). In some cases ALS patients suffer from double and blurred vision.

Some additional neurodegenerative diseases associated with optic neuropathy as described in Pelak, V. S. Ophthalmol. Clin. N. Am. (2004),17:311-320 include Chacot-Marie-Tooth Disease, Mucopolysaccharidoses, Adrenoleukodystrophy, Niemann-Pick disease, Krabbe's disease, Pelizaeus-Merzbacher disease, Progressive encephalopathy, edema, hypsarrhythmia and optic atrophy (PEHO).

Traumatic eye injuries occur from incidents such as from being poked in the eye or hit on the head. Depending on the type of trauma, symptoms can include blurred vision, bulging eye, burning, double vision, dry eyes, floaters, light sensitivity and pain or discomfort of the eye or around the eye. Other occurrences that can occur include swelling, a pupil that is dilated or unresponsive to light, vision loss, limited eye or lid movement or ptosis (drooping eyelids). An estimated 10 to 13 percent of wounded Iraq war veterans have sustained direct, penetrating eye damage, typically as a result of modern weaponry that unleashes an explosive cascade of fragments. Some of these service members are suffering from injuries that stem from trauma in the brain affecting the visual neurological pathways.

Traumatic Optic Neuropathy (TON) refers to an acute injury of the optic nerve secondary to trauma. The optic nerve axons may be damaged either directly or indirectly and the visual loss may be partial or complete. An indirect injury to the optic nerve typically occurs from the transmission of forces to the optic canal from blunt head trauma. This is in contrast to direct TON, which results from an anatomical disruption of the optic nerve fibers from penetrating orbital trauma, bone fragments within the optic canal, or nerve sheath hematomas.

Patients undergoing corneal transplant or stem cell transplant of eye cells may also undergo trauma.

Acute orbital compartment syndrome is a rare but treatable complication of increased pressure within the confined orbital space as a result of facial trauma. The condition presents with recognizable physical findings and progressive visual deficit.

Vitamin E is a generic description for all tocopherol and tocotrienol derivatives. The tocopherols have a phytyl chain. The tocotrienols have a superficially similar chain, but with three double bonds at positions 3′, 7′, and 11′; these double bonds provide the tocotrienols with very different biological and physical properties compared to the tocopherols. Both tocopherols and tocotrienols have four isomers, designated as alpha, beta, gamma and delta, which differ by the number and position of methyl groups on the chroman ring.

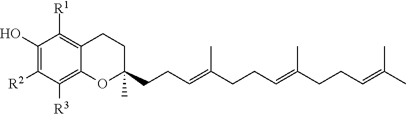

| | | | |
|---|---|---|---|
| Alpha-Tocotrienol | R¹ = CH₃ | R² = CH₃ | R³ = CH₃ |
| Beta-Tocotrienol | R¹ = CH₃ | R² = H | R³ = CH₃ |
| Gamma-Tocotrienol | R¹ = H | R² = CH₃ | R³ = CH₃ |
| Delta-Tocotrienol | R¹ = H | R² = H | R³ = CH₃ |

Although tocopherols and tocotrienols are often described within the generic Vitamin E description as equivalent, it has been lately observed that they have widely varying degrees of biological effectiveness. For example, Tanito et al., “Distribution of Tocopherols and Tocotrienols to Rat Ocular Tissues after Topical Ophthalmic Administration,” Lipids, (2004) Vol 39, No. 5:469-474, showed that the concentration of alpha-tocotrienol increased markedly in every tissue to which it was administered, however no significant increase was observed in the case of alpha-tocopherol. The superior neuroprotective activity of tocotrienol over tocopherol is described by Khanna S, et al. “Neuroprotective properties of the natural vitamin E alpha-tocotrienol”, Stroke (2005) 36 (10): 2258-64.

The use of Vitamin E tocopheryl derivatives, not the use of tocotrienol derivatives, in ophthalmic compositions has been described in U.S. Pat. No. 5,886,030; however, these derivatives are used to increase the aqueous solubility of certain poorly soluble ophthalmic agents, and not as the active compound in the prevention, amelioration, treatment or suppression of ophthalmic neurodegenerative diseases. It is however envisioned within the spirit of the invention that vitamin E tocopheryl derivatives might be included in the ocular formulations to provide additional comfort and non-irritability to said formulations.

The use of tocotrienols for the inhibition of the pathogen Chlamydia is described in patent publication US 2006/0241174. This publication claims the mode of application of Vitamin E tocochromanol in the treatment of Chlamydia with eye drops.

### SUMMARY OF THE INVENTION

The invention relates to a method for preventing, reducing, ameliorating or treating ophthalmic disorders or for stopping the progression of or reversing the loss of vision in a patient in need of such treatment, comprising the topical, periocular, or intraocular application of an ophthalmic formulation, wherein the ophthalmic formulation comprises a therapeutically effective amount of one or more agents selected from the group consisting of alpha-tocotrienol, beta-tocotrienol, gamma-tocotrienol, delta-tocotrienol, or esters or mixtures thereof.

The invention relates to a method for preventing, reducing, ameliorating or treating ophthalmic disorders associated with a neurodegenerative disease or trauma, comprising the topical, periocular, or intraocular application of an ophthalmic formulation wherein the ophthalmic formulation comprises a therapeutically effective amount of one or more agents selected from the group consisting of alpha-tocotrienol, beta-tocotrienol, gamma-tocotrienol, delta-tocotrienol, or esters or mixtures thereof. In some embodiments the formulation comprises alpha-tocotrienol or esters or mixtures thereof; in one particular embodiment the formulation comprises alpha-tocotrienol. In some embodiments the formulation additionally comprises an ophthalmically acceptable vehicle.

In another embodiment, the invention relates to a method for preventing, reducing, ameliorating or treating ophthalmic disorders, particularly ophthalmic disorders associated with neurodegenerative diseases or trauma, comprising administering to a patient in need of such treatment, a topical, periocular, or intraocular application of an ophthalmic formulation comprising a pharmaceutically effective amount of a tocotrienol, wherein the amount of tocotrienol prevents, reduces, ameliorates or treats the ophthalmic disorder.

In another embodiment, the invention relates to a method for preventing, reducing, ameliorating or treating ophthalmic disorders, particularly ophthalmic disorders associated with neurodegenerative diseases or trauma, comprising administering to a patient in need of such treatment, a topical, periocular, or intraocular application of an ophthalmic formulation comprising a pharmaceutically effective amount of a tocotrienol, wherein the amount of tocotrienol is a dose of between about 1 mg and about 1000 mg per day and wherein the tocotrienol prevents, reduces, ameliorates or treats the ophthalmic disorder.

In another embodiment, or any of the foregoing embodiments, the invention relates to a method for preventing, reducing, ameliorating or treating ophthalmic disorders, particularly ophthalmic disorders associated with neurodegenerative diseases or trauma, comprising the topical, periocular, or intraocular application of an ophthalmic formulation wherein the ophthalmic formulation comprises alpha-tocotrienol having a purity of 75% to 99% or of about 75% to about 99%.

In another embodiment, the invention relates to a method for preventing, reducing, ameliorating or treating ophthalmic disorders associated with mitochondrial myopathies including but not limited to the group consisting of inherited mitochondrial diseases; including but not limited to Leber's Hereditary Optic Neuropathy (LHON); Dominant Optic Atrophy (DOA); Chronic Progressive External Ophthalmoplegia (CPEO); Spinocerebellar ataxia (SCA), also called Machado-Joseph disease; Leigh's Syndrome; Friedreich's ataxia (FRDA); Mitochondrial myopathy, Encephalopathy, Lactacidosis, and Stroke (MELAS); Myoclonic Epilepsy with Ragged Red Fibers (MERRF); Kearns-Sayre Syndrome (KSS); Co-Enzyme Q10 (CoQ10) Deficiency; Complex I deficiency; Complex II deficiency; Complex III deficiency; Complex IV deficiency; and Complex V deficiency, comprising administering to a patient in need of such treatment a formulation comprising a therapeutically effective amount of one or more agents selected from the group consisting of alpha-tocotrienol, beta-tocotrienol, gamma-tocotrienol, delta-tocotrienol, or esters or mixtures thereof. In other embodiments, the invention relates to a method for preventing, reducing, ameliorating or treating ophthalmic disorders associated with mitochondrial myopathies including but not limited to Leber's Hereditary Optic Neuropathy (LHON), Dominant Optic Atrophy (DOA), and Chronic Progressive External Ophthalmoplegia (CPEO), comprising administering to a patient in need of such treatment a formulation comprising a therapeutically effective amount of one or more agents selected from the group consisting of alpha-tocotrienol, beta-tocotrienol, gamma-tocotrienol, delta-tocotrienol, or esters or mixtures thereof. In some embodiments, the formulation comprises alpha-tocotrienol or esters or mixtures thereof; in one particular embodiment the formulation comprises alpha-tocotrienol. In some embodiments, the formulation additionally comprises an ophthalmically acceptable vehicle.

In another embodiment, the invention relates to a method for preventing, reducing, ameliorating or treating ophthalmic disorders associated with neurodegenerative diseases or trauma including but not limited to glaucoma, diabetic retinopathy, macular degeneration including age-related macular degeneration and juvenile macular degeneration, Alzheimer's, Progressive Supranuclear palsy (PSP), Parkinson Disease (PD) and other Parkinson-like diseases (called Parkinsonisms), Amyotrophic lateral sclerosis (ALS), Chacot-Marie-Tooth Disease, Mucopolysaccharidoses, Adrenoleukodystrophy, Niemann-Pick disease, Krabbe's disease, Pelizaeus-Merzbacher disease, and Progressive encephalopathy, edema, hypsarrhythmia and optic atrophy (PEHO), comprising administering to a patient in need of such treatment a formulation comprising a therapeutically effective amount of one or more agents selected from the group consisting of alpha-tocotrienol, beta-tocotrienol, gamma-tocotrienol, delta-tocotrienol, or esters or mixtures thereof. In some embodiments, the formulation comprises alpha-tocotrienol or esters or mixtures thereof; in one particular embodiment the formulation comprises alpha-tocotrienol. In some embodiments, the formulation additionally comprises an ophthalmically acceptable vehicle.

In another embodiment, the invention relates to a method for preventing, reducing, ameliorating or treating ophthalmic disorders associated with trauma including but not limited to retinal ischemia, acute retinopathies associated with trauma, post-surgical complications, the damage associated with laser therapy including photodynamic therapy (PDT), traumatic optic neuropathy (TON), surgical light induced iatrogenic retinopathy, corneal transplants and stem cell transplant of eye cells, comprising administering to a patient in need of such treatment a formulation comprising a therapeutically effective amount of one or more agents selected from the group consisting of alpha-tocotrienol, beta-tocotrienol, gamma-tocotrienol, delta-tocotrienol, or esters or mixtures thereof. In some embodiments, the formulation comprises alpha-tocotrienol or esters or mixtures thereof; in one particular embodiment the formulation comprises alpha-tocotrienol. In some embodiments the formulation additionally comprises an ophthalmically acceptable vehicle.

In another embodiment, the invention relates to a method for preventing, reducing, ameliorating or treating ophthalmic disorders associated with an overlap syndrome, such as the overlap syndrome characterized by clinical features of both myoclonus epilepsy ragged-red fibers (MERRF) and Kearns-Sayre syndrome (KSS), which is due to a mitochondrial DNA (mtDNA) mutation at nucleotide 3255 (G3255A) of the tRNA^{Leu(UUR)} gene, comprising administering to a patient in need of such treatment a formulation comprising one or more agents selected from the group consisting of alpha-tocotrienol, beta-tocotrienol, gamma-tocotrienol, delta-tocotrienol, or esters or mixtures thereof. In one embodiment, the formulation comprises an ophthalmically effective amount of alpha-tocotrienol. In other embodiments, the formulation additionally comprises a pharmaceutically acceptable vehicle.

In another aspect, the invention relates to the topical, periocular, or intraocular use of alpha-tocotrienol, beta-tocotrienol, gamma-tocotrienol, delta-tocotrienol, or esters or mixtures thereof, to prevent, reduce, ameliorate or treat ophthalmic disorders in individuals in need of such treatment. In one embodiment, the invention relates to the topical, periocular, or intraocular use of alpha-tocotrienol or an ester thereof, to prevent, reduce, ameliorate or treat ophthalmic disorders in individuals in need of such treatment. In another embodiment, the invention relates to the topical, periocular, or intraocular use of alpha-tocotrienol, to prevent, reduce, ameliorate or treat ophthalmic disorders in individuals in need of such treatment. In another embodiment, the invention relates to the topical, periocular, or intraocular use of alpha-tocotrienol having a purity of 75% to 99% or of about 75% to about 99%, to prevent, reduce, ameliorate or treat ophthalmic disorders in individuals in need of such treatment. In one embodiment, the invention relates to the topical, periocular, or intraocular use of beta-tocotrienol or an ester thereof, to prevent, reduce, ameliorate or treat ophthalmic disorders in individuals in need of such treatment. In another embodiment, the invention relates to the topical, periocular, or intraocular use of beta-tocotrienol, to prevent, reduce, ameliorate or treat ophthalmic disorders in individuals in need of such treatment. In one embodiment, the invention relates to the topical, periocular, or intraocular use of gamma-tocotrienol or an ester thereof, to prevent, reduce, ameliorate or treat ophthalmic disorders in individuals in need of such treatment. In another embodiment, the invention relates to the topical, periocular, or intraocular use of gamma-tocotrienol, to prevent, reduce, ameliorate or treat ophthalmic disorders in individuals in need of such treatment. In one embodiment, the invention relates to the topical, periocular, or intraocular use of delta-tocotrienol or an ester thereof, to prevent, reduce, ameliorate or treat ophthalmic disorders in individuals in need of such treatment. In one embodiment, the invention relates to the topical, periocular, or intraocular use of delta-tocotrienol, to prevent, reduce, ameliorate or treat ophthalmic disorders in individuals in need of such treatment.

In some embodiments, the tocotrienol is an ophthalmically acceptable tocotrienol ester. In some embodiments the tocotrienol ester is selected from the group consisting of an acetic acid ester, a nicotinic acid ester, a linoleic acid ester, a palmitic acid ester, and a succinic acid ester. In other embodiments the tocotrienol is PEGylated; in one such embodiment, the tocotrienol is a tocotrienol polyethylene glycol succinate derivative.

In one embodiment, the invention relates to the topical, periocular, or intraocular use of tocotrienols or mixtures thereof, to prevent, reduce, ameliorate or treat ophthalmic disorders or to stop the progression of or reverse the loss of vision of a patient suffering from or at risk of mitochondrial myopathies. In other embodiments, the mitochondrial myopathy is selected from the group consisting of inherited mitochondrial diseases including but not limited to Leber's Hereditary Optic Neuropathy (LHON); Dominant Optic Atrophy (DOA); Chronic Progressive External Ophthalmoplegia (CPEO); Spinocerebellar ataxia (SCA), also called Machado-Joseph disease; Leigh's Syndrome; Friedreich's ataxia (FRDA); Mitochondrial myopathy, Encephalopathy, Lactacidosis, and Stroke (MELAS); Myoclonic Epilepsy with Ragged Red Fibers (MERRF); Kearns-Sayre Syndrome (KSS); Co-Enzyme Q10 (CoQ10) Deficiency; Complex I deficiency; Complex II deficiency; Complex III deficiency; Complex IV deficiency; and Complex V deficiency.

In one embodiment, the invention relates to the topical, periocular, or intraocular use of tocotrienol esters or mixtures thereof, to prevent, reduce, ameliorate or treat ophthalmic disorders or to stop the progression of or reverse the loss of vision of a patient suffering from or at risk of mitochondrial myopathies. In other embodiments, the mitochondrial myopathy is selected from the group consisting of inherited mitochondrial diseases including but not limited to Leber's Hereditary Optic Neuropathy (LHON); Dominant Optic Atrophy (DOA); Chronic Progressive External Ophthalmoplegia (CPEO); Spinocerebellar ataxia (SCA), also called Machado-Joseph disease; Leigh's Syndrome; Friedreich's ataxia (FRDA); Mitochondrial myopathy, Encephalopathy, Lactacidosis, and Stroke (MELAS); Myoclonic Epilepsy with Ragged Red Fibers (MERRF); Kearns-Sayre Syndrome (KSS); Co-Enzyme Q10 (CoQ10) Deficiency; Complex I deficiency; Complex II deficiency; Complex III deficiency; Complex IV deficiency; and Complex V deficiency.

In some embodiments, the mitochondrial disorder associated with ophthalmic disorders or vision loss is selected from the group consisting of inherited mitochondrial diseases including but not limited to Leber's Hereditary Optic Neuropathy (LHON); Dominant Optic Atrophy (DOA); Chronic Progressive External Ophthalmoplegia (CPEO); Spinocerebellar ataxia (SCA), also called Machado-Joseph disease; Myoclonic Epilepsy with Ragged Red Fibers (MERRF); Mitochondrial Myopathy, Encephalopathy, Lactacidosis, Stroke (MELAS); Leigh's Disease; Kearns-Sayre Syndrome (KSS); and Friedreich's Ataxia (FRDA).

In other embodiments of the invention, including any of the foregoing embodiments, the mitochondrial disorder is Leber's Hereditary Optic Neuropathy (LHON). In another embodiment of the invention, including any of the foregoing embodiments, the mitochondrial disorder is Dominant Optic Atrophy (DOA). In another embodiment of the invention, the mitochondrial disorder is Chronic Progressive External Ophthalmoplegia (CPEO). In another embodiment of the invention, the mitochondrial disorder is spinocerebellar ataxia (SCA), also called Machado-Joseph disease. In another embodiment of the invention, including any of the foregoing embodiments, the mitochondrial disorder is Friedreich's ataxia (FRDA). In another embodiment of the invention, the mitochondrial disorder is Mitochondrial Myopathy, Encephalopathy, Lactacidosis, and Stroke (MELAS). In another embodiment of the invention, the mitochondrial disorder is Kearns-Sayre Syndrome (KSS). In another embodiment of the invention, including any of the foregoing embodiments, the mitochondrial disorder is Leigh's syndrome. In another embodiment of the invention, the mitochondrial disorder is Myoclonic Epilepsy with Ragged Red Fibers (MERRF).

In another embodiment including any of the foregoing embodiments, the neurodegenerative disorder associated with ophthalmic disorders or vision loss is selected from the group consisting of glaucoma, diabetic retinopathy, macular degeneration including age-related macular degeneration and juvenile macular degeneration, Alzheimer's, Progressive Supranuclear palsy (PSP), Parkinson Disease (PD) and other Parkinson-like diseases (called Parkinsonisms), Amyotrophic lateral sclerosis (ALS), Chacot-Marie-Tooth Disease, Mucopolysaccharidoses, Adrenoleukodystrophy, Niemann-Pick disease, Krabbe's disease, Pelizaeus-Merzbacher disease, and Progressive encephalopathy, edema, hypsarrhythmia and optic atrophy (PEHO).

In another embodiment of the invention, the neurodegenerative disorder is Alzheimer's disease. In another embodiment of the invention the neurodegenerative disorder is Progressive Supranuclear Palsy (PSP). In another embodiment of the invention, the neurodegenerative disorder is Parkinson Disease (PD) and other Parkinson-like diseases (called Parkinsonisms). In another embodiment of the invention, the neurodegenerative disorder is Amyotrophic Lateral Sclerosis (ALS).

In another embodiment of the invention, the patient is suffering from glaucoma. In other embodiments of the invention, the patient is suffering from Primary Open-Angle Glaucoma (POAG). In other embodiments of the invention, the patient is suffering from Closed-Angle Glaucoma.

In another embodiment of the invention, the patient is suffering from diabetic retinopathy (DR).

In another embodiment of the invention, the patient is suffering from macular degeneration (MD). In some embodiments of the invention, the patient is suffering from age-related macular degeneration (AMD). In other embodiments of the invention the patient is suffering from juvenile macular degeneration (JMD).

In another embodiment, the invention relates to the topical, periocular, or intraocular use of tocotrienols or mixtures thereof to ameliorate or treat ophthalmic disorders or to stop the progression of or reverse the loss of vision of a patient suffering from traumatic eye injuries. In some embodiments the traumatic injury is Traumatic Optic Neuropathy (TON). In other embodiments, the invention relates to the topical, periocular, or intraocular use of tocotrienols for the amelioration or treatment of patients undergoing corneal transplants or stem cell transplant of eye cells.

In other embodiments, the invention relates to the topical, periocular, or intraocular use of tocotrienols or esters or mixtures thereof for the amelioration or treatment of patients with acute retinopathies associated with trauma, post-surgical complications, the damage associated with laser therapy including photodynamic therapy (PDT), traumatic optic neuropathy (TON), surgical light induced iatrogenic retinopathy, corneal transplants and stem cell transplant of eye cells.

In another embodiment, including any of the foregoing embodiments, the use of the tocotrienols or esters or mixtures thereof is by topical administration. In another embodiment the use of the tocotrienols is by topical administration of eye drops. In another embodiment the use of the tocotrienols is by topical administration of irrigating solution. In another embodiment, including any of the foregoing embodiments the use of the tocotrienols is by periocular administration. In another embodiment, including any of the foregoing embodiments the use of the tocotrienols is by intraocular administration.

In another embodiment, including any of the foregoing embodiments, the tocotrienol formulations are useful as prophylactics to prevent the occurrence of ophthalmic neurodegenerative diseases and loss of vision.

In another embodiment, the invention relates to the use of an ophthalmic formulation comprising a therapeutically effective amount of one or more ophthalmic agents selected from the group consisting of alpha-tocotrienol, beta-tocotrienol, gamma-tocotrienol, delta-tocotrienol, or esters or mixtures thereof and an ophthalmically acceptable vehicle, for the prevention of ophthalmic disorders or vision loss associated with neurodegenerative diseases or trauma. In some embodiments, the ophthalmic agent comprises alpha-tocotrienol or esters thereof; in one particular embodiment the ophthalmic agent comprises alpha-tocotrienol. In some embodiments, the patient is suffering from or at risk of, Leber's Hereditary Optic Neuropathy (LHON), Dominant Optic Atrophy (DOA) or Chronic Progressive External Ophthalmoplegia (CPEO).

In another embodiment, the invention relates to the use of an ophthalmic formulation comprising a therapeutically effective amount of one or more ophthalmic agents selected from the group consisting of alpha-tocotrienol, beta-tocotrienol, gamma-tocotrienol, delta-tocotrienol, or esters or mixtures thereof and an ophthalmically acceptable vehicle, for the reduction of ophthalmic disorders or vision loss associated with neurodegenerative diseases or trauma. In some embodiments, the ophthalmic agent comprises alpha-tocotrienol or esters thereof; in one particular embodiment the ophthalmic agent comprises alpha-tocotrienol. In some embodiments, the patient is suffering from or at risk of, Leber's Hereditary Optic Neuropathy (LHON), Dominant Optic Atrophy (DOA) or Chronic Progressive External Ophthalmoplegia (CPEO).

In another embodiment, the invention relates to the use of an ophthalmic formulation comprising a therapeutically effective amount of one or more ophthalmic agents selected from the group consisting of alpha-tocotrienol, beta-tocotrienol, gamma-tocotrienol, delta-tocotrienol, or esters or mixtures thereof and an ophthalmically acceptable vehicle, for the amelioration or treatment of ophthalmic disorders or vision loss associated with neurodegenerative diseases or trauma. In some embodiments, the ophthalmic agent comprises alpha-tocotrienol or esters thereof; in one particular embodiment the ophthalmic agent comprises alpha-tocotrienol. In some embodiments, the patient is suffering from or at risk of, Leber's Hereditary Optic Neuropathy (LHON), Dominant Optic Atrophy (DOA) or Chronic Progressive External Ophthalmoplegia (CPEO).

In another embodiment, the invention relates to the use of an ophthalmic formulation comprising a therapeutically effective amount of one or more ophthalmic agents selected from the group consisting of alpha-tocotrienol, beta-tocotrienol, gamma-tocotrienol, delta-tocotrienol, or esters or mixtures thereof and an ophthalmically acceptable vehicle, for the treatment of ophthalmic disorders or vision loss associated with neurodegenerative diseases or trauma. In some embodiments, the ophthalmic agent comprises alpha-tocotrienol or esters thereof; in one particular embodiment the ophthalmic agent comprises alpha-tocotrienol. In some embodiments, the patient is suffering from or at risk of Leber's Hereditary Optic Neuropathy (LHON), Dominant Optic Atrophy (DOA) or Chronic Progressive External Ophthalmoplegia (CPEO).

In another aspect the invention relates to a topical ophthalmic formulation wherein the therapeutically effective ophthalmic agent is selected from the group consisting of alpha-tocotrienol, beta-tocotrienol, gamma-tocotrienol, delta-tocotrienol, or esters or mixtures thereof. In some embodiments, the invention relates to a topical ophthalmic formulation wherein the therapeutically effective ophthalmic agent is selected from the group consisting of alpha-tocotrienol or esters or mixtures thereof. In some particular embodiments, the therapeutically effective ophthalmic agent is alpha-tocotrienol having a purity of 75% to 99% or of about 75% to about 99%.

In some embodiments the ophthalmic formulation is administered topically as eye drops. In another embodiment the ophthalmic formulation is administered topically as an irrigating solution. In another embodiment, the ophthalmic formulation is administered periocularly. In another embodiment, the ophthalmic formulation is administered intraocularly.

In another aspect, the invention relates to a topical ophthalmic formulation beneficial for the protection against, reduction, amelioration or treatment of ophthalmic disorders or vision loss, said formulation comprising a therapeutically effective amount of one or more agents selected from the group consisting of alpha-tocotrienol, beta-tocotrienol, gamma-tocotrienol, delta-tocotrienol, or esters or mixtures thereof. In some embodiments the formulation comprises alpha-tocotrienol, or esters or mixtures thereof; in one particular embodiment the formulation comprises alpha-tocotrienol. In some embodiments the topical ophthalmic formulation additionally comprises an ophthalmically acceptable vehicle.

In another embodiment, the invention relates to a topical ophthalmic formulation beneficial in a patient suffering from or at risk of ophthalmic disorders or vision loss associated with neurodegenerative diseases or trauma, said formulation comprising a therapeutically effective amount of alpha-tocotrienol or an alpha-tocotrienol ester and an ophthalmically acceptable vehicle. In some embodiments the formulation comprises alpha-tocotrienol, or esters or mixtures thereof; in one particular embodiment the formulation comprises alpha-tocotrienol. In some embodiments the topical ophthalmic formulation additionally comprises an ophthalmically acceptable vehicle.

In another embodiment, the invention relates to a topical ophthalmic formulation beneficial to a patient suffering from or at risk of ophthalmic disorders or vision loss associated with mitochondrial myopathies, including but not limited to Leber's Hereditary Optic Neuropathy (LHON); Dominant Optic Atrophy (DOA); Chronic Progressive External Ophthalmoplegia (CPEO); Spinocerebellar ataxia (SCA), also called Machado-Joseph disease; Leigh's Syndrome; Friedreich's ataxia (FRDA); Mitochondrial myopathy, Encephalopathy, Lactacidosis, and Stroke (MELAS); Myoclonic Epilepsy with Ragged Red Fibers (MERRF); Kearns-Sayre Syndrome (KSS); Co-Enzyme Q10 (CoQ10) Deficiency; Complex I deficiency; Complex II deficiency; Complex III deficiency; Complex IV deficiency; and Complex V deficiency, said formulation comprising a therapeutically effective amount of alpha-tocotrienol or an alpha-tocotrienol ester. In some embodiments the formulation comprises alpha-tocotrienol. In some embodiments the topical ophthalmic formulation additionally comprises an ophthalmically acceptable vehicle.

In some embodiments, the patient is suffering from or at risk of, Leber's Hereditary Optic Neuropathy (LHON), Dominant Optic Atrophy (DOA) or Chronic Progressive External Ophthalmoplegia (CPEO).

In another embodiment, the invention relates to a topical ophthalmic formulation beneficial to a patient suffering from or at risk of ophthalmic disorders or vision loss associated with neurodegenerative diseases including but not limited to glaucoma, diabetic retinopathy, macular degeneration including age-related macular degeneration and juvenile macular degeneration, Alzheimer's, Progressive Supranuclear palsy (PSP), Parkinson Disease (PD) and other Parkinson-like diseases (called Parkinsonisms), Amyotrophic lateral sclerosis (ALS), Chacot-Marie-Tooth Disease, Mucopolysaccharidoses, Adrenoleukodystrophy, Niemann-Pick disease, Krabbe's disease, Pelizaeus-Merzbacher disease, and Progressive encephalopathy, edema, hypsarrhythmia and optic atrophy (PEHO), said formulation comprising a therapeutically effective amount of alpha-tocotrienol or alpha-tocotrienol ester. In some embodiments the formulation comprises alpha-tocotrienol. In some embodiments the topical ophthalmic formulation additionally comprises an ophthalmically acceptable vehicle.

In another embodiment, the invention relates to a topical ophthalmic formulation beneficial in a patient suffering from or at risk of ophthalmic disorders or vision loss associated with trauma including but not limited to retinal ischemia, acute retinopathies associated with trauma, post-surgical complications, the damage associated with laser therapy including photodynamic therapy (PDT), traumatic optic neuropathy (TON), surgical light induced iatrogenic retinopathy, corneal transplants and stem cell transplant of eye cells.

### DETAILED DESCRIPTION OF THE INVENTION

The present invention discloses compounds, formulations, methods and kits for use in patients. A patient is a mammal, preferably a human.

“Treating” a disease with the compounds and methods discussed herein is defined as administering one or more of the compounds discussed herein, with or without additional therapeutic agents, in order to reduce or eliminate either the disease or one or more symptoms of the disease, or to retard the progression of the disease or of one or more symptoms of the disease, or to reduce the severity of the disease or of one or more symptoms of the disease. “Suppression” of a disease with the compounds and methods discussed herein is defined as administering one or more of the compounds discussed herein, with or without additional therapeutic agents, in order to suppress the clinical manifestation of the disease, or to suppress the manifestation of adverse symptoms of the disease. The distinction between treatment and suppression is that treatment occurs after adverse symptoms of the disease are manifest in a subject, while suppression occurs before adverse symptoms of the disease are manifest in a subject. Suppression may be partial, substantially total, or total.

Because some of the diseases are due to genetic mutations, genetic screening can be used to identify patients at risk of the disease. The compounds and methods of the invention can then be administered to asymptomatic patients at risk of developing the clinical symptoms of the disease, in order to suppress the appearance of any adverse symptoms.

“Therapeutic use” of the compounds discussed herein is defined as using one or more of the compounds discussed herein to treat or suppress a disease, as defined above. A “therapeutically effective amount” of a compound is an amount of the compound, which, when administered to a subject, is sufficient to reduce or eliminate either a disease or one or more symptoms of a disease, or to retard the progression of a disease or of one or more symptoms of a disease, or to reduce the severity of a disease or of one or more symptoms of a disease, or to suppress the clinical manifestation of a disease, or to suppress the manifestation of adverse symptoms of a disease. A therapeutically effective amount can be given in one or more administrations.

The active component of the formulation of the present invention is selected from alpha-tocotrienol, beta-tocotrienol, gamma-tocotrienol, delta-tocotrienol, or esters or mixtures thereof. In one embodiment, the formulation of the present invention comprises alpha-tocotrienol as the active component. In other embodiments, the formulations of the present invention comprise one or more tocotrienols in an ophthalmically acceptable vehicle for topical, periocular, or intraocular administration, and in other embodiments, the formulations of the present invention comprise alpha-tocotrienol in an ophthalmically acceptable vehicle.

In some embodiments, the active components are ophthalmically acceptable tocotrienol esters or mixtures thereof. In some embodiments, the tocotrienol esters are selected from tocotrienol acetate, tocotrienol succinate, tocotrienol phosphate, tocotrienol aspartate, tocotrienol glutamate, tocotrienol palmitate, tocotrienol nicotinate, and polyethoxylated tocotrienol. In some embodiments, the active components are ophthalmically acceptable alpha-tocotrienol esters. In some embodiments, the alpha-tocotrienol esters are selected from alpha-tocotrienol acetate, alpha-tocotrienol succinate, alpha-tocotrienol phosphate, alpha-tocotrienol aspartate, alpha-tocotrienol glutamate, alpha-tocotrienol palmitate, alpha-tocotrienol nicotinate, and polyethoxylated alpha-tocotrienol.

Tocotrienols belong to the vitamin E family and differ from the tocopherols in the chemical nature of the side chain or tail and in their properties that are clearly distinct from those of the tocopherols. Tocotrienols are fat-soluble, water-insoluble oils and modulate several mechanisms associated with the aging process and aging-related diseases.

Tocotrienols have been shown to promote healthy cholesterol levels, neuroprotective, anti-oxidant activity and anti-cancer effects that are often not exhibited by tocopherols. The main commercial sources of tocotrienols are rice bran oil, palm oil, and annatto bean. Annatto tocotrienol extracts contain no alpha-tocotrienol, 90% of delta-tocotrienol, 10% of gamma-tocotrienol, and no tocopherols. Rice bran extract contains less than 2% alpha-tocotrienol. 51.6% gamma-tocotrienol and 48% tocopherols. Palm oil extract contains 22.1% alpha-tocotrienol, 10% delta-tocotrienol 45.7% gamma-tocotrienol and 21.8% tocopherols. Tocotrienols are often used in small quantities as antioxidants to prevent deformulation and enhance the stability of other components in formulations, but in the present invention tocotrienols are used as one of the essential components of the topical, periocular, or intraocular formulation for the prevention, reduction and amelioration of ophthalmic disorders related to neurodegenerative diseases or trauma. That is, the tocotrienols are used in a therapeutically effective amount for the prevention, reduction and amelioration of ophthalmic disorders related to neurodegenerative diseases or trauma.

The formulations of the present invention particularly comprise alpha-tocotrienol which can be produced synthetically or derived from one of the commercial sources mentioned above. A preferred process for the production of essentially pure alpha-tocotrienol has been described in co-owned U.S. provisional application U.S. A No. 61/197,585 filed Oct. 28, 2008 titled “Process for Enrichment and Isolation of alpha-Tocotrienol from Natural Extracts,” in co-owned U.S. patent application Ser. No. 12/606,923 filed Oct. 27, 2009, and in co-owned International Patent Application PCT/US2009/062212 filed Oct. 27, 2009.

Syntheses of various members of the tocotrienol family in the d,l- or (RS)-form have been published, see for example Schudel et al, Helv. Chim. Acta (1963) 46, 2517-2526; H. Mayer et al, Helv. Chim. Acta (1967) 50, 1376-11393; H.-J. Kabbe et al, Synthesis (1978), 888-889; M. Kajiwara et al, Heterocycles (1980) 14, 1995-1998; S. Urano et al, Chem. Pharm. Bull. (1983) 31, 4341-4345, Pearce et al, J. Med Chem. (1992), 35, 3595-3606 and Pearce et al, J. Med. Chem. (1994). 37, 526-541. None of these reported processes lead to the natural form of the tocotrienols, but rather produce racemic mixtures. Syntheses of natural form d-tocotrienols have been published. See for example. J. Scott et al, Helv. Chim. Acta (1976) 59, 290-306, Sato et al (Japanese Patent 63063674); Sato et al (Japanese Patent No. JP 01-233278) and Couladouros et al (U.S. Pat. No. 7,038,067).

While synthetic and natural tocopherols are readily available in the market, the natural tocotrienol supply is limited, and generally comprises a mixture of tocotrienols. Crude palm oil which is rich in tocotrienols (800-1500 ppm) offers a potential source of natural tocotrienols. Carotech, Malaysia is able to extract and concentrate tocotrienols from crude palm oil, by a process patented in U.S. Pat. No. 5,157,132. Tocomin®-50 typically comprises about 25.32% mixed tocotrienols (7.00% alpha-tocotrienol, 14.42% gamma-tocotrienol, 3.30% delta-tocotrienol and 0.6% beta-tocotrienol), 6.90% alpha-tocopherol and other phytonutrients such as plant squalene, phytosterols, co-enzyme Q10 and mixed carotenoids.

Other methods for isolation or enrichment of tocotrienol from certain plant oils and plant oil by-products have been described in the literature. For some examples of such isolation and purification processes, see for instance Top A. G. et al, U.S. Pat. No. 5,190,618; Lane R. et al, U.S. Pat. No. 6,239,171; Bellafiore, L. et al U.S. Pat. No. 6,395,915; May, C. Y. et al, U.S. Pat. No. 6,656,358; Jacobs, L. et al, U.S. Pat. No. 6,838,104; Sumner, C. et al Int. Pat. Pub. WO 99/38860, or Jacobs, L. Int. Pat. Pub. WO 02/500054.

The formulations administered according to the present invention may also include various other ingredients, including but not limited to surfactants, tonicity agents, buffers, preservatives, co-solvents and viscosity building agents.

According to some embodiments of the methods of the present invention, a formulation comprising one or more tocotrienols, preferably alpha-tocotrienol, and a ophthalmically acceptable carrier for topical ophthalmic administration or implantation into the conjunctival sac or anterior chamber of the eye is administered to a patient in need thereof. The compositions are formulated in accordance with methods known in the art for the particular route of administration desired.

The formulations administered topically, periocularly, or intraocularly comprise a ophthalmically effective amount of one or more tocotrienols, preferably alpha-tocotrienol. As used herein, an “ophthalmically effective amount” is one which is sufficient to reduce or eliminate signs or symptoms of the ophthalmic disorders described herein. Generally, for formulations intended to be administered topically to the eye in the form of eye drops or eye ointments, the total amount of the tocotrienol will be 0.001 to 1.0% (w/w). When applied as eye drops, 1-2 drops (approximately 20-45 μl each) of such formulations will be administered from once to several times per day.

The preferred route of administration is topical. The compounds of the present invention can be administered as solutions, suspensions, or emulsions (dispersions) in an ophthalmically acceptable vehicle. An “ophthalmically acceptable” component, as used herein, refers to a component which will not cause any significant ocular damage or ocular discomfort at the intended concentration and over the time of intended use. Solubilizers and stabilizers should be non-reactive. An “ophthalmically acceptable vehicle” refers to any substance or combination of substances which are non-reactive with the compounds and suitable for administration to a patient. Suitable vehicles may be non-aqueous liquid media including the physiologically acceptable oils such as silicone oil, USP mineral oil, white oil, poly(ethylene-glycol), a polyethoxylated castor oil and vegetable oils, for example corn oil, peanut oil, or the like. Other suitable vehicles may be aqueous or oil-in-water solutions suitable for topical application to the patient's eyes. These vehicles may be preferred based on ease of formulation, as well as a patient's ability to easily administer such compositions by means of instilling one to two drops of the solutions in the affected eyes. The compositions may also be suspensions, viscous or semi-viscous gels, or other types of solid or semi-solid compositions and fat bases, such as natural wax e.g. white bees wax, carnauba wax, wool wax (wool fat), purified lanolin, anhydrous lanolin; petroleum wax e.g. solid paraffin, microcrystalline wax; hydrocarbons e.g. liquid paraffin, white petrolatum, yellow petrolatum; or combinations thereof. The compositions may be applied by use of the hands or an applicator such as a wipe, a contact lens, a dropper or a spray. The compounds and formulations for use in the present invention can also be administered using a contact lens-based bioactive agent delivery system, such as those described in U.S. Pat. Appl. Pub. No. 2009/0060981.

The compositions administered according to the present invention may also include various other ingredients, including but not limited to surfactants, tonicity agents, buffers, preservatives, co-solvents and viscosity building agents.

Various tonicity agents may be employed to adjust the tonicity of the formulation, preferably to that of natural tears for ophthalmic formulations. For example, sodium chloride, potassium chloride, magnesium chloride, calcium chloride, dextrose and/or mannitol may be added to the formulation to approximate physiological tonicity. Such an amount of tonicity agent will vary, depending on the particular agent to be added. In general, however, the formulations will have a tonicity agent in an amount sufficient to cause the final formulation to have an ophthalmically acceptable osmolality (generally about 200-400 mOsm/kg).

An appropriate buffer system (e.g., sodium phosphate, sodium acetate, sodium citrate, sodium borate or boric acid) may be added to the formulations to prevent pH drift under storage conditions. The particular concentration will vary, depending on the agent employed. Preferably, however, the buffer will be chosen to maintain a target pH within the range of pH 6-7.5.

Compositions formulated for the treatment of ophthalmic disorders associated with neurodegenerative diseases and disorders may also comprise aqueous carriers designed to provide immediate, short-term relief of dry eye-type conditions. Such carriers can be formulated as a phospholipid carrier or an artificial tears carrier, or mixtures of both. As used herein, “phospholipid carrier” and “artificial tears carrier” refer to aqueous formulations which: (i) comprise one or more phospholipids (in the case of phospholipid carriers) or other compounds, which lubricate, “wet,” approximate the consistency of endogenous tears, aid in natural tear build-up, or otherwise provide temporary relief of dry eye symptoms and conditions upon ocular administration; (ii) are safe; and (iii) provide the appropriate delivery vehicle for the topical administration of an effective amount of one or more of the specified compounds of the invention. Examples or artificial tears compositions useful as artificial tears carriers include, but are not limited to, commercial products, such as Tears Naturale®, Tears Naturale II®, Tears Naturale Free® and Bion Tears ®. (Alcon Laboratories, Inc., Fort Worth, Tex.). Examples of phospholipid carrier formulations include those disclosed in U.S. Pat. No. 4,804,539 (Guo et al), U.S. Pat. No. 4,883,658 (Holly), U.S. Pat. No. 4,914,088 (Glonek), U.S. Pat. No. 5,075,104 (Gressel et al), U.S. Pat. No. 5,278,151 (Korb et al), U.S. Pat. No. 5,294,607 (Glonek et al), U.S. Pat. No. 5,371,108 (Korb et al), U.S. Pat. No. 5,578,586 (Glonek et al); the foregoing patents are incorporated herein by reference to the extent they disclose phospholipid compositions useful as phospholipid carriers of the present invention.

Other compounds designed to lubricate, “wet,” approximate the consistency of endogenous tears, aid in natural tear build-up, or otherwise provide temporary relief of dry eye symptoms and conditions upon ocular administration the eye are known in the art. Such compounds may enhance the viscosity of the formulation, and include, but are not limited to: monomeric polyols, such as, glycerol, propylene glycol, ethylene glycol; polymeric polyols, such as, polyethylene glycol, hydroxypropylmethyl cellulose, carboxy methyl cellulose sodium, hydroxypropyl cellulose, dextrans, such as, dextran 70; water soluble proteins, such as gelatin; and vinyl polymers, such as, polyvinyl alcohol, polyvinylpyrrolidone, povidone and carbomers.

Other compounds may also be added to the ophthalmic formulations of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers. In general, the phospholipid carrier or artificial tears carrier formulations will exhibit a viscosity of 1 to 400 centipoises.

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

The compounds for use in the current invention can also be administered via periocular administration, and may be formulated in solutions or suspensions for periocular administration. The formulations of the present invention may be administered periocularly following traumatic events involving the retina and optic nerve head tissues, or prior to or during ophthalmic surgery to prevent damage or injury. Formulations useful for periocular administration will generally be periocular injection formulations or surgical irrigating solutions. Periocular administration refers to administration to tissues near the eye, such as administration to the tissues or spaces surrounding the eyeball and within the orbit. Periocular administration can take place by injection, deposit, or any other mode of placement. Periocular routes of administration include, but are not limited to, subconjunctival, suprachoroidal, juxtascleral, posterior juxtascleral, sub-Tenon, posterior sub-Tenon, retrobulbar, peribulbar, or laterobulbar delivery. Raghava et al., Expert Opin. Drug Deliv. 1(1):99-114 (2004); Ghate et al. Investigative Ophthalmology and Visual Science, 48 (5): 2230 (2007); Karl G. Csaky, Retina Today, pp. 32-35 (March/April 2007); WO 2009/023877; and EP 1611879 describe various routes of periocular administration.

The tocotrienol compositions of the present invention may be formulated in solutions or suspensions for intraocular administration. The compositions of the present invention may be administered intraocularly following traumatic events involving the retina and optic nerve head tissues or prior to or during ophthalmic surgery to prevent damage or injury. Compositions useful for intraocular administration will generally be intraocular injection compositions or surgical irrigating solutions.

The tocotrienol compositions can also be formulated in an ocular irrigating solution used during ophthalmic surgery to treat retinal or optic nerve head damage resulting from trauma due to injury or prevent damage resulting from the invasive nature of the surgery.

In general, the doses utilized for the above described purposes will vary, but will be in an effective amount to prevent, reduce or ameliorate retina or optic nerve head neuropathy. As used herein, “ophthalmically effective amount” or ‘therapeutically effective amount” refers to that amount of active agent which prevents, reduces or ameliorates retina or optic nerve head neuropathy. The tocotrienols will generally be contained in the topical, periocular, or intraocular formulations contemplated herein in an amount of from about 0.001 to about 10.0% weight/volume (“% w/v”). Preferred concentrations will range from about 0.1 to about 5.0% w/v. Topical formulations will generally be delivered to the eye one to six times a day, at the discretion of a skilled clinician.

The formulations of the present invention may contain additional pharmaceutically active agents or may be dosed concurrently with other pharmaceutical compositions. For example, when treating a mammal for the prevention, reduction, treatment or amelioration or treatment of glaucomatous retinopathy, the formulations of the present invention may contain additional “anti-glaucoma” agents or may be dosed concurrently or sequentially with anti-glaucoma agent compositions. Examples of anti-glaucoma agents include: prostaglandins or prostanoids, carbonic anhydrase inhibitors, beta-adrenergic agonists and antagonists, alpha-adrenergic agonists or other anti-glaucoma agents known to those skilled in the art.

### Monitoring Treatment Efficacy

### Optical Coherence Tomography (OCT):

OCT is a non-invasive technology used for imaging the retina, the multi-layered sensory tissue lining the back of the eye. OCT, the first instrument to allow doctors to see cross-sectional images of the retina, is revolutionizing the early detection and treatment of eye conditions such as macular holes, pre-retinal membranes, macular swelling and even optic nerve damage.

Retinal thickness may also be measured using other devices such as the Retinal Thickness Analyzer (RTA; Talia Technology, Ltd., Mevasseret Zion, Israel) and the Heidelberg Retina Tomograph (HRT; Heidelberg Engineering GmbH, Heidelberg, Germany). Persons skilled in the art will appreciate that the slope of retinal thickness may be calculated over any number of distances, and that the smallest distance is only limited by the resolution of the devices used to practice the methods of the invention.

### Ishihara Color Test

The Ishihara Color test is a test for red-green color deficiencies. The test consists of a number of colored plates, called Ishihara plates, each of which contain a circle of dots appearing randomized in color and size. Within the pattern are dots which form a number visible to those with normal color vision and invisible, or difficult to see, for those with a red-green color vision defect. The full test consists of 38 plates, but the existence of a deficiency is usually clear after a few plates. Testing the first 24 plates gives a more accurate diagnosis of the severity of the color vision defect.

Common plates include a circle of dots in shades of green and light blues with a figure differentiated in shades of brown, or a circle of dots in shades of red, orange and yellow with a figure in shades of green; the first testing for protanopia and the second for deuteranopia.

### Dosages

The compounds used in the methods of the invention can be administered in various amounts. Examples of daily dosages which can be used are an effective amount within the dosage range of about 0.1 mg/kg to about 300 mg/kg body weight, or within about 0.1 mg/kg to about 100 mg/kg body weight, or within about 0.1 mg/kg to about 80 mg/kg body weight, or within about 0.1 mg/kg to about 50 mg/kg body weight, or within about 0.1 mg/kg to about 30 mg/kg body weight, or within about 0.1 mg/kg to about 10 mg/kg body weight, or within about 1.0 mg/kg to about 80 mg/kg body weight, or within about 1.0 mg/kg to about 50 mg/kg body weight, or within about 1.0 mg/kg to about 30 mg/kg body weight, or within about 1.0 mg/kg to about 10 mg/kg body weight, or within about 10 mg/kg to about 80 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight, or about or up to about 1, about or up to about 5, about or up to about 10, about or up to about 15, about or up to about 20, about or up to about 25, about or up to about 30, about or up to about 40, about or up to about 50, about or up to about 60, about or up to about 70, about or up to about 75, about or up to about 80, about or up to about 90, about or up to about 100, about or up to about 125, about or up to about 150, about or up to about 175, about or up to about 200, about or up to about 225, about or up to about 250, about or up to about 275, about or up to about 300, about or up to about 325, about or up to about 350, about or up to about 375, about or up to about 400, about or up to about 425, about or up to about 450, about or up to about 500, about or up to about 550, about or up to about 600, about or up to about 650, about or up to about 700, about or up to about 750, about or up to about 800, about or up to about 850, about or up to about 900, about or up to about 950, or about or up to about 1000 mg total. The compound(s) may be administered in a single daily dose, or the total daily dosage may be administered in divided dosage of two, three or four times daily. Treatment should be continuous with the tocotrienol or tocotrienol ester being administered daily at the above-mentioned dosages for a definite or an indefinite duration, as prescribed by the medical team.

### Kits

The invention also provides articles of manufacture and kits containing materials useful for treating ophthalmic diseases. The article of manufacture comprises a container with a label. Suitable containers include, for example, bottles, vials, and test tubes. The containers may be formed from a variety of materials such as glass or plastic. The container holds a compound selected from alpha-tocotrienol, beta-tocotrienol, gamma-tocotrienol, and delta-tocotrienol, or a composition comprising an active agent selected from alpha-tocotrienol, beta-tocotrienol, gamma-tocotrienol, and delta-tocotrienol. In one embodiment, the compound is alpha-tocotrienol. In one embodiment, the active agent is alpha-tocotrienol. The label on the container indicates that the composition is used for treating ophthalmic diseases, and may also indicate directions for use in treatment.

The invention also provides kits comprising any one or more of a compound selected from alpha-tocotrienol, beta-tocotrienol, gamma-tocotrienol, and delta-tocotrienol, or a composition comprising an active agent selected from alpha-tocotrienol, beta-tocotrienol, gamma-tocotrienol, and delta-tocotrienol. In some embodiments, the kit of the invention comprises the container described above, which holds a compound selected from alpha-tocotrienol, beta-tocotrienol, gamma-tocotrienol, and delta-tocotrienol, or a composition comprising an active agent selected from alpha-tocotrienol, beta-tocotrienol, gamma-tocotrienol, and delta-tocotrienol. In other embodiments, the kit of the invention comprises the container described above, which holds a compound selected from alpha-tocotrienol, beta-tocotrienol, gamma-tocotrienol, and delta-tocotrienol, or a composition comprising an active agent selected from alpha-tocotrienol, beta-tocotrienol, gamma-tocotrienol, and delta-tocotrienol, and a second container comprising a vehicle for the compound or composition, such as one or more vegetable-derived oils, such as sesame oil, and/or one or more animal-derived oils, and/or one or more fish-derived oils. In other embodiments, the kit of the invention comprises the container described above, which holds a compound selected from alpha-tocotrienol, beta-tocotrienol, gamma-tocotrienol, and delta-tocotrienol, or a composition comprising an active agent selected from alpha-tocotrienol, beta-tocotrienol, gamma-tocotrienol, and delta-tocotrienol, where the compound or composition has been pre-mixed with a vehicle for the compound or composition, such as one or more vegetable-derived oils, such as sesame oil, and/or one or more animal-derived oils, and/or one or more fish-derived oils. The kits may further include other materials desirable from a commercial and user standpoint, including other vehicles, buffers, diluents, filters, needles, syringes, and package inserts with instructions for performing any of the methods described herein for treatment of ophthalmic diseases.

In other aspects, the kits may be used for any of the methods described herein, including, for example, to prevent, reduce, ameliorate, or treat ophthalmic disorders or to stop the progression of or reverse the loss of vision in individual with optic myopathies as for example LHON and DOA.

Treatment of a Patient Diagnosed with LHON

A patient with Leber's Hereditary Optic Neuropathy is treated with alpha-tocotrienol. Alpha-tocotrienol is administered to the patient via topical administration; the drug is mixed with a suitable carrier for topical ophthalmic administration. The drug-carrier mixture is instilled in the eyes three times daily on an ongoing basis for at least three months.

While being treated with alpha-tocotrienol, the patient's medical team monitors the patient's eyes for any signs of improvement or signs of worsening of the disease by measuring visual acuity, color vision, vision field and OCT.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A method for preventing, reducing, ameliorating, or treating ophthalmic disorders or for stopping the progression of loss of vision or reversing the loss of vision in a patient in need of such treatment, comprising administering to the patient a therapeutically effective amount of a topical, periocular, or intraocular ophthalmic formulation, wherein the ophthalmic formulation comprises one or more agents selected from the group consisting of alpha-tocotrienol, beta-tocotrienol, gamma-tocotrienol, delta-tocotrienol, or esters or mixtures thereof.

2. The method according to claim 1, wherein the ophthalmic formulation comprises alpha-tocotrienol or an ophthalmically acceptable ester thereof.

3. The method according to claim 1, wherein the ophthalmic formulation comprises alpha-tocotrienol.

4. The method according to claim 3, wherein the alpha-tocotrienol has a purity of about 75 to about 99%.

5. The method according to claim 3, wherein the ophthalmic formulation is administered topically.

6. The method according to claim 5, wherein the formulation is administered in eye drops.

7. The method according to claim 5, wherein the ophthalmic formulation is an irrigating solution.

8. The method according to claim 3, wherein the ophthalmic formulation is administered periocularly.

9. The method according to claim 3, wherein the ophthalmic formulation is administered intraocularly.

10. The method according to claim 1, wherein the patient in need of such treatment is suffering from or at risk of an ophthalmic disorder associated with a disorder selected from the group of inherited mitochondrial diseases selected from Leber's Hereditary Optic Neuropathy (LHON);
Dominant Optic Atrophy (DOA); Chronic Progressive External Ophthalmoplegia (CPEO); Spinocerebellar ataxia (SCA), also called Machado-Joseph disease; Leigh's Syndrome; Friedreich's ataxia (FRDA);
Mitochondrial Myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; Complex V deficiency; neurodegenerative diseases; Parkinson's disease; Alzheimer's disease; Amyotrophic Lateral Sclerosis (ALS); motor neuron diseases; Huntington's Disease; age-associated diseases; glaucoma; disorders of the outer retina; macular degeneration; age related macular degeneration, juvenile macular degeneration; retinal ischemia; acute retinopathies associated with trauma; post-surgical complications, the damage associated with laser therapy including photodynamic therapy (PDT); traumatic optic neuropathy (TON); the damage associated with surgical light induced iatrogenic retinopathy; the damage associated with corneal transplants and the damage associated with stem cell transplant of eye cells.

11. The method according to claim 2, wherein the patient in need of such treatment is suffering from or at risk of an ophthalmic disorder associated with a disorder selected from the group of inherited mitochondrial diseases selected from Leber's Hereditary Optic Neuropathy (LHON); Dominant Optic Atrophy (DOA); Chronic Progressive External Ophthalmoplegia (CPEO); Spinocerebellar ataxia (SCA), also called Machado-Joseph disease; Leigh's Syndrome; Friedreich's ataxia (FRDA); Mitochondrial Myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; Complex V deficiency; neurodegenerative diseases; Parkinson's disease; Alzheimer's disease; Amyotrophic Lateral Sclerosis (ALS); motor neuron diseases; Huntington's Disease; age-associated diseases; glaucoma; disorders of the outer retina; macular degeneration; age related macular degeneration, juvenile macular degeneration; retinal ischemia; acute retinopathies associated with trauma; post-surgical complications, the damage associated with laser therapy including photodynamic therapy (PDT); traumatic optic neuropathy (TON); the damage associated with surgical light induced iatrogenic retinopathy; the damage associated with corneal transplants and the damage associated with stem cell transplant of eye cells.

12. The method according to claim 3, wherein the patient in need of such treatment is suffering from or at risk of an ophthalmic disorder associated with a disorder selected from the group of inherited mitochondrial diseases selected from Leber's Hereditary Optic Neuropathy (LHON); Dominant Optic Atrophy (DOA); Chronic Progressive External Ophthalmoplegia (CPEO); Spinocerebellar ataxia (SCA), also called Machado-Joseph disease; Leigh's Syndrome; Friedreich's ataxia (FRDA); Mitochondrial Myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; Complex V deficiency; neurodegenerative diseases; Parkinson's disease; Alzheimer's disease; Amyotrophic Lateral Sclerosis (ALS); motor neuron diseases; Huntington's Disease; age-associated diseases; glaucoma; disorders of the outer retina; macular degeneration; age related macular degeneration, juvenile macular degeneration; retinal ischemia; acute retinopathies associated with trauma; post-surgical complications, the damage associated with laser therapy including photodynamic therapy (PDT); traumatic optic neuropathy (TON); the damage associated with surgical light induced iatrogenic retinopathy; the damage associated with corneal transplants and the damage associated with stem cell transplant of eye cells.

13. The method according to claim 1, wherein the ophthalmic disorder is associated with a mitochondrial myopathy selected from the group consisting of inherited mitochondrial diseases selected from Leber's Hereditary Optic Neuropathy (LHON); dominant optic atrophy (DOA); Chronic Progressive External Ophthalmoplegia (CPEO); Spinocerebellar ataxia (SCA), also called Machado-Joseph disease; Myoclonic Epilepsy with Ragged Red Fibers (MERRF); Mitochondrial Myopathy, Encephalopathy, Lactacidosis, Stroke (MELAS); Leigh's Disease; Kearns-Sayre Syndrome (KSS); overlap syndromes; and Friedreich's Ataxia (FRDA).

14. The method according to claim 2, wherein the ophthalmic disorder is associated with a mitochondrial myopathy selected from the group consisting of inherited mitochondrial diseases selected from Leber's Hereditary Optic Neuropathy (LHON); dominant optic atrophy (DOA); Chronic Progressive External Ophthalmoplegia (CPEO); Spinocerebellar ataxia (SCA), also called Machado-Joseph disease; Myoclonic Epilepsy with Ragged Red Fibers (MERRF); Mitochondrial Myopathy, Encephalopathy, Lactacidosis, Stroke (MELAS); Leigh's Disease; Kearns-Sayre Syndrome (KSS); overlap syndromes; and Friedreich's Ataxia (FRDA).

15. The method according to claim 3, wherein the ophthalmic disorder is associated with a mitochondrial myopathy selected from the group consisting of inherited mitochondrial diseases selected from Leber's Hereditary Optic Neuropathy (LHON); dominant optic atrophy (DOA); Chronic Progressive External Ophthalmoplegia (CPEO); Spinocerebellar ataxia (SCA), also called Machado-Joseph disease; Myoclonic Epilepsy with Ragged Red Fibers (MERRF); Mitochondrial Myopathy, Encephalopathy, Lactacidosis, Stroke (MELAS); Leigh's Disease; Kearns-Sayre Syndrome (KSS); overlap syndromes; and Friedreich's Ataxia (FRDA).

16. The method according to claim 15, wherein the ophthalmic disorder is Leber's Hereditary Optic Neuropathy (LHON) or dominant optic atrophy (DOA).

17. The method according to claim 15, wherein the ophthalmic disorder is associated with Friedreich's Ataxia (FRDA).

18. The method according to claim 1, where the ophthalmic disorder is selected from the group consisting of glaucoma, diabetic retinopathy, macular degeneration; age-related macular degeneration; juvenile macular degeneration, or wherein the ophthalmic disorder is associated with Alzheimer's, Progressive Supranuclear Palsy (PSP), Parkinson Disease (PD) and other Parkinson-like diseases (called Parkinsonisms), Amyotrophic lateral sclerosis (ALS), Chacot-Marie-Tooth Disease, Mucopolysaccharidoses, Adrenoleukodystrophy, Niemann-Pick disease, Krabbe's disease, Pelizaeus-Merzbacher disease, and Progressive Encephalopathy, Edema, Hypsarrhythmia and Optic Atrophy (PEHO).

19. The method according to claim 2, where the ophthalmic disorder is selected from the group consisting of glaucoma, diabetic retinopathy, macular degeneration; age-related macular degeneration; juvenile macular degeneration, or wherein the ophthalmic disorder is associated with Alzheimer's, Progressive Supranuclear Palsy (PSP), Parkinson Disease (PD) and other Parkinson-like diseases (called Parkinsonisms), Amyotrophic lateral sclerosis (ALS), Chacot-Marie-Tooth Disease, Mucopolysaccharidoses, Adrenoleukodystrophy, Niemann-Pick disease, Krabbe's disease, Pelizaeus-Merzbacher disease, and Progressive Encephalopathy, Edema, Hypsarrhythmia and Optic atrophy (PEHO).

20. The method according to claim 3, where the ophthalmic disorder is selected from the group consisting of glaucoma, diabetic retinopathy, macular degeneration; age-related macular degeneration; juvenile macular degeneration, or wherein the ophthalmic disorder is associated with Alzheimer's, Progressive supranuclear palsy (PSP), Parkinson Disease (PD) and other Parkinson-like diseases (called Parkinsonisms), Amyotrophic lateral sclerosis (ALS), Chacot-Marie-Tooth Disease, Mucopolysaccharidoses, Adrenoleukodystrophy, Niemann-Pick disease, Krabbe's disease, Pelizaeus-Merzbacher disease, and Progressive Encephalopathy, Edema, Hypsarrhythmia and Optic Atrophy (PEHO).

21. The method according to claim 20, where the ophthalmic disorder is glaucoma.

22. The method according to claim 20, where the ophthalmic disorder is macular degeneration.

23. The method according to claim 20, where the ophthalmic disorder is diabetic neuropathy.

24-41. (canceled)

42. A topical, periocular, or intraocular ophthalmic formulation beneficial to a patient suffering from or at risk of ophthalmic disorders or vision loss, said formulation comprising a therapeutically effective amount of one or more agents selected from the group consisting of alpha-tocotrienol, beta-tocotrienol, gamma-tocotrienol, delta-tocotrienol, or esters or mixtures thereof.

43. The topical, periocular, or intraocular ophthalmic formulation according to claim 42, comprising a therapeutically effective amount of alpha-tocotrienol or alpha-tocotrienol ester.

44. The topical, periocular, or intraocular ophthalmic formulation according to claim 42, comprising tocotrienol esters selected from tocotrienol acetate, tocotrienol succinate, tocotrienol phosphate, tocotrienol aspartate, tocotrienol glutamate, tocotrienol palmitate, tocotrienol nicotinate, and polyethoxylated tocotrienol.

45-62. (canceled)

63. The method according to claim 5, wherein the patient in need of such treatment is suffering from or at risk of an ophthalmic disorder associated with a disorder selected from the group of inherited mitochondrial diseases selected from Leber's Hereditary Optic Neuropathy (LHON); Dominant Optic Atrophy (DOA); Chronic Progressive External Ophthalmoplegia (CPEO).

64. The method according to claim 8, wherein the patient in need of such treatment is suffering from or at risk of an ophthalmic disorder associated with a disorder selected from the group of inherited mitochondrial diseases selected from Leber's Hereditary Optic Neuropathy (LHON); Dominant Optic Atrophy (DOA); Chronic Progressive External Ophthalmoplegia (CPEO).

65. The method according to claim 9, wherein the patient in need of such treatment is suffering from or at risk of an ophthalmic disorder associated with a disorder selected from the group of inherited mitochondrial diseases selected from Leber's Hereditary Optic Neuropathy (LHON); Dominant Optic Atrophy (DOA); Chronic Progressive External Ophthalmoplegia (CPEO).
